(19) United States
(12) Patent Application Publication
(10) Pub. No.: US 2020/0390688 A1
(43) Pub. Date: Dec. 17, 2020

(54) LONG-ACTING TOPICAL FORMULATION AND METHOD OF USE THEREOF

(71) Applicant: PIEDMONT ANIMAL HEALTH INC., Greensboro, NC (US)

(72) Inventors: Douglas Hepler, Greensboro, NC (US); Neil E. Paulsen, Greensboro, NC (US); Michael S. Daniel, Greensboro, NC (US); Gail L. Dempsey, Greensboro, NC (US)

(21) Appl. No.: 16/900,201

(22) Filed: Jun. 12, 2020

Related U.S. Application Data
(60) Provisional application No. 62/861,621, filed on Jun. 14, 2019.

(51) Int. Cl. A61K 9/00; A61K 47/14; A61K 31/42

(52) U.S. Cl. CPC A61K 9/0014; A61K 31/42; A61K 47/14

(57) ABSTRACT

Provided herein are long-acting, non-aqueous pharmaceutically acceptable compositions of active ingredients for topical administration.

### CROSS REFERENCE TO RELATED APPLICATION

This application claims the benefit of priority to U.S. Provisional Patent Application Ser. No. 62/861,621, filed on Jun. 14, 2019. The entire contents of the foregoing is hereby incorporated by reference in its entirety.

### BACKGROUND OF THE INVENTION

### Field of the Invention

The invention relates generally to long-acting, non-aqueous, topical formulations and more specifically to long-acting formulations especially for use in mammals.

### Background Information

A number of parasites can infest or infect domestic animals especially also companion animals such as cats and dogs. These pests and parasites are of great nuisance to both the animals and their owners.

One such parasite is heartworm. Heartworm infection in cats and dogs is a worldwide clinical problem. Despite improved diagnostic methods, effective preventives, and increasing awareness among veterinary professionals and pet owners, heartworm has yet to be eradicated.

Adult female heartworms release their young, called microfilariae, into an animal's bloodstream. Then, mosquitoes become infected with microfilariae while taking blood meal from the infected animal. During the next 10 to 14 days, the microfilariae mature to the infective larval stage within the mosquito. After that, the mosquito bites another dog, cat or other susceptible animal, and the infective larvae enter through the bite wound. It then takes a little over 6 months for the infective larvae to mature into adult worms which, in dogs, may live for up to 7 years.

Commonly known products for treatment of heartworm include ADVANTAGE MULTI® (moxidectin and imidacloprid), HEARTGARD® (ivermectin and pyrantel), INTERCEPTOR® (milbemycin) and REVOLUTION® (selamectin). However, there are currently no topical products available that adequately treat heartworm along with other parasitic infections, such as fleas and ticks, that provide robust treatment of 2-3 months or greater after a single topical administration.

Therefore, it would be desirable to have compositions which provide prolonged therapeutic relief to a mammal for parasitic infection while minimizing the number of administrations/doses that must be given to the mammal.

### SUMMARY OF THE INVENTION

In one aspect, the disclosure provides a long-acting, non-aqueous, topical pharmaceutically acceptable composition.

In one embodiment, the topical pharmaceutically acceptable composition includes:

a) a pharmaceutically active agent;

b) N-methyl-2-pyrrolidone (NMP);

c) 2-pyrrolidone; and

d) a triglyceride carrier.

In embodiments, the combination of NMP and 2-pyrrolidone are present at about 30.0 to 70.0% w/w. For example, NMP may be present at about 15.0 to 25.0% w/w and 2-pyrrolidone may be present at about 25.0 to 35.0% w/w.

In another embodiment, the topical pharmaceutically acceptable composition includes:

a) a pharmaceutically active agent;

b) benzyl alcohol; and

c) propylene carbonate.

In embodiments, the benzyl alcohol is present at about 50.0 to 90.0% w/w and the propylene carbonate is present at about 5.0 to 30.0% w/w.

In embodiments, the pharmaceutically active agent is present at about 5.0 to 20.0% w/w or about 5.0 to 15.0% w/w or about 10.0% w/w.

In embodiments, the pharmaceutically active agent is a macrolide parasiticide and/or antimicrobial, optionally in combination with an isoxazoline compound. In various embodiments, the macrolide antiparasitic is moxidectin, selamectin, milbemycin, ivermectin, doramectin, emamectin, eprinomectin, doximectin, abimectin, roxithromycin, clarithromycin, tulathromycin, gamithromycin, dirithromycin, fidaxomicin, megalomicin, erythromycin, azithromycin, or combination thereof. In one embodiment, the macrolide parasiticide is moxidectin. In embodiments, the isoxazoline compound is afoxolaner, fluralaner, sarolaner, lotilaner, or a combination thereof. In one embodiment, the composition includes a combination of moxidectin and fluralaner.

In embodiments, the pharmaceutically active agent is moxidectin present at about 5.0 to 20.0% w/w or about 5.0 to 15.0% w/w or about 10.0% w/w.

In certain aspects, an exemplary formulation is as set forth in Table I below, where moxidectin may be substituted or supplemented with any pharmaceutically active agent.

TABLE I
Formulation

| Component | w/w % |
|---|---|
| Moxidectin | 5 |
| Medium chain triglycerides | 44-46 |
| NMP | 20 |
| 2-pyrrolidone | 30 |
| BHT | 0-1 |
| | 100 |

In certain aspects, an exemplary formulation is as set forth in Table II below, where moxidectin may be substituted or supplemented with any pharmaceutically active agent.

TABLE II
Formulation

| Component | w/w % |
|---|---|
| Moxidectin | 15 |
| Medium chain triglycerides | 34-36 |
| NMP | 20 |
| 2-pyrrolidone | 30 |
| BHT | 0-1 |
| | 100 |

In certain aspects, an exemplary formulation is as set forth in Table III below, where moxidectin may be substituted or supplemented with any pharmaceutically active agent.

TABLE III
Formulation

| Component | w/w % |
|---|---|
| Moxidectin | 5 |
| Benzyl alcohol | 79-81 |
| Propylene carbonate | 15 |
| BHT | 0-1 |
| | 100 |

In certain related aspects, an exemplary formulation is as set forth in Table IV below, where moxidectin may be substituted or supplemented with any pharmaceutically active agent.

TABLE IV
Formulation

| Component | w/w % |
|---|---|
| Moxidectin | 15 |
| Benzyl alcohol | 70-72 |
| Propylene carbonate | 14 |
| BHT | 0-1 |
| | 100 |

In certain related aspects, an exemplary formulation is as set forth in Table V below, where moxidectin may be substituted or supplemented with any pharmaceutically active agent.

TABLE V
Formulation

| Component | w/w % |
|---|---|
| Moxidectin | 5-15 |
| Benzyl alcohol | 70-80 |
| Propylene carbonate | 10-20 |
| BHT | 0-2 |
| | 100 |

In certain related aspects, an exemplary formulation is as set forth in Table VI below, where moxidectin may be substituted or supplemented with any pharmaceutically active agent.

TABLE VI
Formulation

| Component | w/w % |
|---|---|
| Moxidectin | 10 |
| Benzyl alcohol | 75-76 |
| Propylene carbonate | 15-16 |
| BHT | 0.5-1.5 |
| | 100 |

In certain embodiments, the pharmaceutically active agent is present in an amount of about 0.25 to 25.0% w/w. In other aspects, the triglycerides are caprylic/capric triglycerides or caprylic triglycerides. In other embodiments, the triglyceride is present in an amount of up to about 40.0% w/w±15.0% w/w. In other aspects, the composition is sterile and formulated for topical administration.

Also provided herein is a method of treating a disease or disorder (e.g., parasitic invention, ectoparasitic infestation) in a subject by administering a formulation of the invention. Surprisingly, a clinically effective amount of the pharmaceutically active agent when topically administered in a formulation of the invention is present in the blood stream of the subject for about 2, 3, 4, 5, 6 months or greater after topical administration. In various embodiments, a single, or multiple pharmaceutically active agents are administered in a single formulation.

The disclosure also provides a method of preventing or treating heartworm in a subject by topically administering a formulation of the invention including a macrolide parasiticide.

Further provided is a method of killing ectoparasites on a subject by topically administering a formulation of the invention including an isoxazoline.

The disclosure also provides a method of killing ectoparasites on a subject and preventing or treating heartworm in the subject by topically administering a formulation of the invention including a macrolide parasiticide and an isoxazoline, such as moxidectin and fluralaner.

In embodiments, the composition of the disclosure is administered at most once every 2, 3, 4, 5 or 6 months and the subject remains substantially heartworm free and free of living ectoparasites for at least 2, 3, 4, 5, 6 months or more after each administration. In embodiments, the formulation kills heartworm within the subject for 3 months or greater and kills ectoparasites on the subject for 3 months or greater upon a single topical administration. In embodiments, the formulation kills heartworm within the subject for 3 months or greater and kills ectoparasites on the subject for 4 months or greater upon a single topical administration. In embodiments, the formulation kills heartworm within the subject for 4 months or greater and kills ectoparasites on the subject for 4 months or greater upon a single topical administration. In embodiments, the formulation kills heartworm within the subject for 5 months or greater and kills ectoparasites on the subject for 5 months or greater upon a single topical administration. In embodiments, the formulation kills heartworm within the subject and kills ectoparasites on the subject for up to 4, 5, 6 months or greater upon a single topical administration.

In embodiments, formulations including moxidectin and/or fluralaner are particularly suited for treatment of canines for heartworm and ectoparasitic infestation.

### BRIEF DESCRIPTION OF THE FIGURES

FIG. 1 is a graphical representation depicting data in one embodiment of the invention.

### DETAILED DESCRIPTION OF THE INVENTION

The following terms, definitions and abbreviations apply. Abbreviations used herein have their conventional meaning within the chemical and biological arts.

The term “subject” refers to mammalian organisms to be treated by the methods of the disclosure. Such organisms include, but are not limited to, companion animals such as domestic dogs and cats. In the context of the disclosure, the term “subject” generally refers to an individual who will receive or who has received treatment described below (e.g., administration of the compositions of the disclosure).

As used herein, a “patient” or “subject” refers to either a human or non-human mammalian animal. Non-human animals include any non-human mammalian animals. Such non-human animals may include, but are not limited to rodents, non-human primates (e.g., monkey and apes), ungulates, ovines, bovines, ruminants, lagomorphs, porcines, caprines, equines, canines, felines, murines, and the like. In certain embodiments of the invention, the animals are mammals. In some embodiments, the animals include, but are not limited to, companion animals such as domestic dogs and cats. In the context of the disclosure, the term “subject” generally refers to an individual who will receive or who has received treatment described below (e.g., administration of a composition of the disclosure).

The term “therapeutically effective amount” means the amount of the compound or pharmaceutical composition that will elicit the biological or medical response of a patient or tissue that is being sought by the researcher, veterinarian, medical doctor or other clinician.

By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the disclosure or pharmaceutical composition to the subject in need of treatment.

The term “about” with respect to a number means that the number includes a range of insignificant variation above and below the number unless otherwise stated; e.g., a value of 1 will be understood to include up to 0.5 to 1.5 and all numbers thereinbetween.

In embodiments, the pharmaceutical compositions of the invention are in the form of a non-aqueous topical solution of an active such as a macrolide and/or an isoxazoline compound, in a carrier including (i) NMP, (ii) 2-pyrrolidone, and (iii) a triglyceride carrier.

In embodiments, the NMP and 2-pyrrolidone are both present in an amount of about 5.0 to 40.0% w/w of the composition, or 10.0 to 40.0% w/w, or 15.0 to 35.0% w/w or 20.0 to 30.0% w/w, such as about 20.0% w/w or 30.0% w/w.

In embodiments, the NMP is present in an amount of about 15.0 to 25.0% w/w of the composition, and 2-pyrrolidone is present in an amount of about 25.0 to 35.0% w/w of the composition, such as about 20.0% w/w and about 30.0% w/w respectively.

As indicated in the example the inventors discovered, that the presently described formulations provide a pharmacokinetic profile in which blood plasma levels of the active are extended beyond 2, 3, 4, 5 month or more, and which are higher than a comparable dose of active administered via subcutaneous injection or oral administration.

This formulation approach provides unexpectedly significant improvement in bioavailability. Hence, similar blood levels of active can be achieved that lead to prolonged effectiveness to ameliorate disease and/or control parasites as compared to subcutaneous injection or oral administration.

In embodiments, the triglyceride is present in an amount of about 5.0 to 70.0% w/w, or 25.0 to 60.0% w/w, 30.0 to 60.0% w/w, or 30.0 to 50.0% w/w. In some embodiments, the triglyceride is caproic acid, caprylic acid, capric acid, lauric acid, myristic acid or any combination thereof. For example, the triglyceride is a medium chain triglyceride, such as caprylic/capric (C8 and/or C10) triglycerides or caprylic (C8) triglycerides. In embodiments, the triglyceride is a mixture of caprylic acid and capric acid wherein the mixture comprises about 40.0 to 85.0% caprylic acid and about 15.0 to 60.0% capric acid, or wherein the mixture comprises about 50.0 to 80.0% caprylic acid and about 20.0 to 50.0% capric acid, or wherein the mixture comprises about 65.0 to 80.0% caprylic acid and about 20.0 to 35.0% capric acid, or wherein the mixture comprises about 50.0 to 65.0% caprylic acid and about 30.0 to 45.0% capric acid. In one embodiment, the triglyceride may be a fatty acid ester emollient, such as a saturated coconut and palm kernel oil-derived caprylic/capric fatty acid mixture with glycerin in a solid form sold under the trademark MIGLYOL™. In another embodiment, the triglyceride may be a fatty acid ester emollient, such as a saturated coconut and palm kernel oil-derived caprylic/capric fatty acid mixture sold under the trademark CAPTEX™, such as CAPTEX™ 8000.

It will be appreciated that the triglyceride used in the composition may be entirely substituted or supplement with a monoglyceride or diglyceride, the fatty acid moieties of which are saturated or unsaturated, preferably saturated, and contain from 6 to 30 carbon atoms. In some embodiments, the fatty acid moieties of the glyceride contain from 18 to 24 carbon atoms, more preferably from 20 to 22 carbon atoms.

The term ‘saturated’ as used herein refers to fatty acid moieties containing only carbon-carbon single bonds, e.g., an alkyl group. The term ‘unsaturated’ as used herein refers to fatty acid moieties containing at least one carbon-carbon double or triple bond (e.g., an alkenyl group, —CH₂═CH₂—, or an alkynyl group, Any alkenyl groups which may be present may exist in either cis or trans geometries. In some embodiments, the fatty acid moieties of the fat are either saturated, or unsaturated with one or more alkenyl groups.

In another embodiment, the pharmaceutical compositions of the invention are in the form of a non-aqueous topical solution of an active such as a macrolide and/or an isoxazoline compound, in a carrier including (i) benzyl alcohol, and (ii) propylene carbonate.

In embodiments, the benzyl alcohol is present in an amount of about 50.0 to 90.0% w/w of the composition, 60.0 to 85.0% w/w, or 60.0 to 80.0% w/w or 70.0 to 80.0% w/w, such as about 70.0, 70.1, 70.2, 70.3, 70.4, 70.5, 70.6, 70.7, 70.8, 70.9 or 80.0% w/w, or about 71.0, 72.0, 73.0, 74.0, 75.0, 76.0, 77.0, 78.0, 79.0 or 80.0% w/w.

In embodiments, the propylene carbonate is present in an amount of about 5.0 to 30.0% w/w, or 10.0 to 30.0% w/w or 10.0 to 20.0% w/w or 12.0 to 20.0% w/w or 12.0 to 18.0% w/w, such as about 12.0, 13.0, 14.0, 15.0, 16.0, 17.0, 18.0, 19.0 or 20.0% w/w.

The composition may also contain excipients. In certain aspects, excipients include ethanol, 2-ethoxy (2-ethoxy) ethanol, ethyl oleate, ethyl acetate, ethyl benzoate, benzyl alcohol, glycerol, polyethylene glycol 200, polyethylene glycol 300, polyethylene glycol 400, benzyl benzoate, isopropyl myristate, isopropyl alcohol, 2-pyrrolidone, DMSO, polyvinylpyrrolidone (e.g., PVP K17), propylene carbonate, glycofurol, N-methylpyrrolidone, propylene glycol, acetone, methyl acetate, methyl ethyl ketone, dimethylformamide, dimethylsulfoxide, tetrahydrofuran, caprolactam, decylmethylsulfoxide, tetrahydrofuran, caprolactam, decylmethylsulfoxide, and oleic acid, 1-dodecylazacycloheptan-2-one.

It will be understood that benzyl alcohol may be substituted with another alcohol (e.g., ethanol) which may be present in an amount of about 50.0 to 90.0% w/w of the composition, 60.0 to 85.0% w/w, or 60.0 to 80.0% w/w or 70.0 to 80.0% w/w, such as about 70.0, 70.1, 70.2, 70.3, 70.4, 70.5, 70.6, 70.7, 70.8, 70.9 or 80.0% w/w, or about 71.0, 72.0, 73.0, 74.0, 75.0, 76.0, 77.0, 78.0, 79.0 or 80.0% w/w.

In various embodiments, an alcohol for use in the pharmaceutically acceptable composition of the invention includes one or more alcohols and/or glycols. Such alcohols are pharmaceutically acceptable and are generally liquids at about room temperature, approximately 20° C. By way of illustration, an alcohol or glycol for use in the composition of the invention may include one or more of propylene glycol, ethanol, 2-(2-ethoxyethoxy)ethanol (Transcutol®), benzyl alcohol, glycerol, polyethylene glycol 200, polyethylene glycol 300, polyethylene glycol 400 and the like.

Also, while the pharmaceutically active agent may be in its hydrated form, no water is added to the composition during or after mixture. As such, the composition described herein is substantially non-aqueous, for example, the composition has less than about 3.0, 2.5, 2.0, 1.5, 1.0, 0.9, 0.8, 0.7, 0.6, 0.5, 0.4, 0.3, 0.2, 0.1, 0.5 or 0.1% w/w of an aqueous substance, such as water.

Any or all of the components of the composition may be included in their dehydrated form or their anhydrous form.

An exemplary formulation is as set forth in Table VII below, where moxidectin may be substituted or supplemented with any pharmaceutically active agent, such as fluralaner.

TABLE VII
Formulation

| Component | w/w % |
|---|---|
| Moxidectin | 1-20 |
| Medium chain triglycerides | 30-50 |
| NMP | 15-25 |
| 2-pyrrolidone | 25-35 |
| BHT | 0-1 |
| | 100 |

An exemplary formulation is as set forth in Table VIII below, where moxidectin may be substituted or supplemented with any pharmaceutically active agent.

TABLE VIII
Formulation

| Component | w/w % |
|---|---|
| Moxidectin | 1-20 |
| Benzyl alcohol | 65-85 |
| Propylene carbonate | 11-18 |
| BHT | 0-1 |
| | 100 |

An exemplary formulation is as set forth in Table IX below, where fluralaner may be substituted or supplemented with any pharmaceutically active agent.

TABLE IX
Formulation

| Component | w/w % |
|---|---|
| Fluralaner | 1-20 |
| Medium chain triglycerides | 30-50 |
| NMP | 15-25 |
| 2-pyrrolidone | 25-35 |
| BHT | 0-1 |
| | 100 |

An exemplary formulation is as set forth in Table X below, where fluralaner may be substituted or supplemented with any pharmaceutically active agent.

TABLE X
Formulation

| Component | w/w % |
|---|---|
| Fluralaner | 1-20 |
| Benzyl alcohol | 65-85 |
| Propylene carbonate | 11-18 |
| BHT | 0-1 |
| | 100 |

An exemplary formulation is as set forth in Table XI below, where the pharmaceutically active agent may be substituted or supplemented with any pharmaceutically active agent.

TABLE XI
Formulation

| Component | w/w % |
|---|---|
| Moxidectin and Fluralaner | 1-20 |
| Medium chain triglycerides | 30-50 |
| NMP | 15-25 |
| 2-pyrrolidone | 25-35 |
| BHT | 0-1 |
| | 100 |

An exemplary formulation is as set forth in Table XII below, where the pharmaceutically active agent may be substituted or supplemented with any pharmaceutically active agent.

TABLE XII
Formulation

| Component | w/w % |
|---|---|
| Moxidectin and Fluralaner | 1-20 |
| Benzyl alcohol | 65-85 |
| Propylene carbonate | 11-18 |
| BHT | 0-1 |
| | 100 |

In some embodiments, the composition includes inert ingredients such as antioxidants or preservatives. Antioxidants such as a propyl gallate, BHA (butylated hydroxy anisole), BHT (butylated hydroxy toluene), MTG (monothioglycerol), tri-ethyl citrate, citric acid, TBHQ (tert-butyl hydroquinone) and the like may be added to the present formulation. The antioxidants are generally added to the formulation in amounts of from about 0.01 to about 2.0% (w/w). In certain embodiments, antioxidants are present in an amount of about 0.01 to 2.0%, 0.05 to 2.0%, 0.5 to 2.0% or 0.5 to 1.5%. For example, in embodiments, the composition includes MTG and/or citric acid in an amount of up to, or about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% w/w. In embodiments, the composition includes BHT and/or propyl galate in an amount of up to, or about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% w/w.

Preservatives such as the parabens (methylparaben and/or propylparaben) are suitably used in the formulation in amounts ranging from about 0.01 to about 2.0 w/w.

The formulation of the present invention may be prepared without addition of water to the mixture during any step of the process.

The disclosure provides pharmaceutical compositions comprising at least one pharmaceutically active agent in an amount effective for treating a disease or disorder, and a pharmaceutically acceptable vehicle. The pharmaceutically active agents may be hydrated; e.g., a monohydrate or dihydrate form of the molecule.

A suitable pharmaceutically active agent for use in the formulations described herein is an active pharmaceutical ingredient or a combination of a plurality of active ingredients. Such active pharmaceutical agents include, by way of illustration only, antimicrobials, parasiticides and anthelmintics.

In embodiments, the pharmaceutically active agent is both a parasiticide and anthelmintic.

As such, the disclosure provides compositions comprising at least one pharmaceutically active agent in an amount effective for treating a disease or disorder (such as a parasitic infection, microbial infection, and/or ectoparasitic infestation), and a pharmaceutically acceptable vehicle.

In embodiments, a pharmaceutically active agent for use in the formulations described herein is a macrolide parasiticide and/or antimicrobial. Macrolides may include, but are not limited to moxidectin, selamectin, milbemycin, ivermectin, doramectin, emamectin, eprinomectin, doximectin, abimectin, roxithromycin, clarithromycin, tulathromycin, gamithromycin, dirithromycin, fidaxomicin, megalomicin, erythromycin, azithromycin, or combination thereof. The active agents are typically hydrated; e.g., a monohydrate or dehydrate form of the molecule.

In embodiments, a pharmaceutically active agent for use in the formulations described herein is an isoxazoline.

Isoxazolines may include any isoxazoline known in the art. Isoxazoline compounds and their use as parasiticide are described, for example, in US patent application No. US 2007/0066617, and International Patent applications WO 2007/079162, WO 2009/002809, WO 2009/024541, WO 2009/003075, WO2009/080250, WO 2010/070068, WO 2010/079077, WO 2011/075591 and WO 2011/124998, the disclosures of which, as well as the references cited herein, are incorporated by reference. This class of compounds is known to possess excellent activity against ectoparasites such as ticks and fleas.

The isoxazoline compounds may exist in various isomeric forms. A reference to an isoxazoline compound always includes all possible isomeric forms of such compound. Unless otherwise stated, a compound structure that does not indicate a particular conformation is intended to encompass compositions of all the possible conformational isomers of the compound, as well as compositions comprising fewer than all the possible conformational isomers. In some embodiments, the compound is a chiral compound. In some embodiments, the compound is a non-chiral compound.

Isoxazoline compounds be prepared according to one or other of the processes described e.g. in Patent Applications US 2007/0066617, WO 2007/079162, WO 2009/002809, WO 2009/080250, WO 2010/070068, WO 2010/079077, 2011/075591 and WO 2011/124998 or any other process coming within the competence of a person skilled in the art who is an expert in chemical synthesis. For the chemical preparation of the products of the invention, a person skilled in the art is regarded as having at his disposal, inter alia, the entire contents of “Chemical Abstracts” and of the documents which are cited therein.

In embodiments of the composition according to the disclosure, the isoxazoline is one or more selected from the group consisting of: fluralaner, afoxolaner, lotilaner, sarolaner, (Z)-4-[5-(3,5-Dichlorophenyl)-5-trifluoromethyl-4,5-dihydroisoxazol-3-yl]-N-[(methoxyimino)methyl]-2-methylbenzamide (CAS RN: 928789-76-8), 4-[5-(3,5-dichlorophenyl)-5-(trifluoromethyl)-4H-isoxazol-3-yl]-2-methyl-N-(thietan-3-yl)benzamide (CAS RN: 1164267-94-0), which was disclosed in WO 2009/0080250, and 5-[5-(3,5-Dichlorophenyl)-4,5-dihydro-5-(trifluoromethyl)-3-isoxazolyl]-3-methyl-N-[2-oxo-2-[(2,2,2-trifluoroethyl)amino]ethyl]-2-thiophenecarboxamide (CAS RN: 1231754-09-8), which was disclosed in WO 2010/070068.

The pharmaceutically active compounds of the disclosure may also be formulated into compositions as natural or salt forms. Pharmaceutically acceptable non-toxic salts include the base addition salts (formed with free carboxyl or other anionic groups), which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure include amine salts formed by the protonation of an amino group with inorganic acids such as hydrochloric acid, hydrobromic acid, hydroiodic acid, sulfuric acid, phosphoric acid, and the like. Salts of the disclosure may also include amine salts formed by the protonation of an amino group with suitable organic acids, such as p-toluenesulfonic acid, acetic acid, and the like.

Additional excipients which are contemplated for use in the practice of the disclosure are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs, hydrates, and solvates of the compounds are included in the disclosure. It should be noted that while the hydrate molecules will contribute water to the pharmaceutical composition, it is envisioned that no other water source be included.

The composition may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a carrier suitable for administration via an intended route, specifically, topical administration. In the pharmaceutical composition, the active compound is included in an amount sufficient to produce the desired effect upon the process or condition of diseases.

For the compositions of the invention, the pharmaceutically active agent need only be administered by single application for an entire course of treatment to clinically resolve or control a disease or disorder. However, the pharmaceutically active agent may be administered by a series of applications, such as 1, 2, 3, 4, 5, 6, 7, 8, 9, 10 or more applications as necessary over a duration to clinically resolve a disease or disorder. In respects, “clinically resolve” or “control” may be measured by reference to the clinically significant and measurable presence of the active in the animal's bloodstream (at least about 1.0 ng/ml) for the requisite period of time, which may be greater than 2, 3, 4, 5 or 6 months from a single application. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition.

Similarly, it is anticipated that the formulations of the disclosure achieve at least 75, 80, 85, 90, 91, 92, 93, 94, 95, 96, 97, 98, 99 or 100% cure rate of the disease or disorder upon single application. It is expected that patients administered the formulations will show at least 75, 80, 85, 90, 91, 92, 93, 94, 95, 96, 97, 98, 99 or 100% cure within 1, 2, 3, 4, 5, 6, 7, 8, 9, 10, 11, 12, 13, 14, 15, 16, 17, 18, 19, 20 or 21 days of administration.

As used herein, “cure rate” refers to clinical efficacy at resolving or controlling the disease or disorder, such as parasitic infection or infestation. In embodiments, the disease or disorder is resolved with an efficacy greater than about 75, 80, 85, 90, 91, 92, 93, 94, 95, 96, 97, 98, 99 or up to 100%, within a duration of less than 1, 2, 3, 4, 5, 6, 7, 8, 9, 10, 11, 12, 13, 14, 15, 16, 17, 18, 19, 20 or 21 days after a single administration.

In one embodiment the composition of the invention is intended for use for controlling a parasitic insect, acarid and/or helminth, especially parasitic insect and/or acarid infestation. The term “controlling a parasitic insect- and/or acarid infestation” refers to preventing, reducing or eliminating an infestation by such parasites on animals preferably by killing the insects and/or acarids or nematode parasites within hours or days.

The term “parasitic insect- and acarid” refers to ectoparasites e.g. insect and acarine pests that commonly infest or infect animals. Examples of such ectoparasites include the egg, larval, pupal, nymphal and adult stages of lice, fleas, mosquitoes, mites, ticks, and biting or nuisance fly species. Especially important are fleas and ticks, especially their adult stages.

Examples of invertebrate parasitic pests controlled by administering the topical formulation of this invention to an animal to be protected include ectoparasites (arthropods, acarines, etc) and endoparasites (helminths, e.g., nematodes, trematodes, cestodes, acanthocephalans, etc.).

In particular, the formulations of this invention are effective against ectoparasites including: flies such as Haematobia (Lyperosia) irritans (horn fly), Stomoxys calcitrans (stable fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus instestinalis, Gastrophilus haemorrhoidalis and Gastrophilus naslis; lice such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; mites such as Psoroptes spp., Sarcoptes scabei, Chorioptes bovis, Demodex equi, Cheyletiella spp., Notoedres cati, Trombicula spp. and Otodectes cyanotis (ear mites); ticks such as Ixodes spp., Boophilus spp., Rhipicephalus spp., Amblyomma spp., Dermacentor spp., Hyalomma spp. and Haemaphysalis spp.; and fleas such as Ctenocephalides felis (cat flea) and Ctenocephalides canis (dog flea).

In embodiments, an appropriate active concentration level will generally be about 0.1 to about 300.0 mg/ml, such as, for example, about 0.25 to 300.0 mg/ml, 1.0 to 300.0 mg/ml, 5.0 to 300.0 mg/ml, 5.0 to 250.0 mg/ml, 5.0 to 200.0 mg/ml, 5.0 to 150.0 mg/ml, 10.0 to 200.0 mg/ml, including 1.0, 10.0, 25.0, 50.0, 75.0, 100.0, 125.0, 150.0, 175.0, 200.0, 225.0, 250.0, 275.0 and 300.0 mg/ml (as well as all other intermediate dosages) all in a single dosage form.

In embodiments, an appropriate active concentration level will generally be about 0.01 to about 500.0 mg/ml or about 0.1 to about 250.0 mg/ml, such as, for example, about 0.25 to 500.0 mg/ml, 1.0 to 400.0 mg/ml, 5.0 to 250.0 mg/ml, 1.0 to 100.0 mg/ml, 5.0 to 150.0 mg/ml, 10.0 to 250.0 mg/ml, 10.0 to 200.0 mg/ml, 15.0 to 250.0 mg/ml or 15.0 to 200.0 mg/ml (including all intermediate dosages) all in a single dosage form.

In embodiments, an appropriate active concentration level will generally be about 0.1 to about 30.0 mg/ml or about 0.1 to about 25.0 mg/ml, such as, for example, about 0.25 to 30.0 mg/ml, 1.0 to 25.0 mg/ml, 5.0 to 25.0 mg/ml, 1.0 to 10.0 mg/ml, 5.0 to 15.0 mg/ml, 10.0 to 25.0 mg/ml, 10.0 to 20.0 mg/ml, 15.0 to 25.0 mg/ml or 15.0 to 20.0 mg/ml (including all intermediate dosages) all in a single dosage form.

The formulations of the invention are particularly useful in mammals, especially companion animals, and most especially cats and dogs.

The following examples are provided to further illustrate the embodiments of the present invention, but are not intended to limit the scope of the invention. While they are typical of those that might be used, other procedures, methodologies, or techniques known to those skilled in the art may alternatively be used.

### Example I

### Formulations and Pharmacokinetics

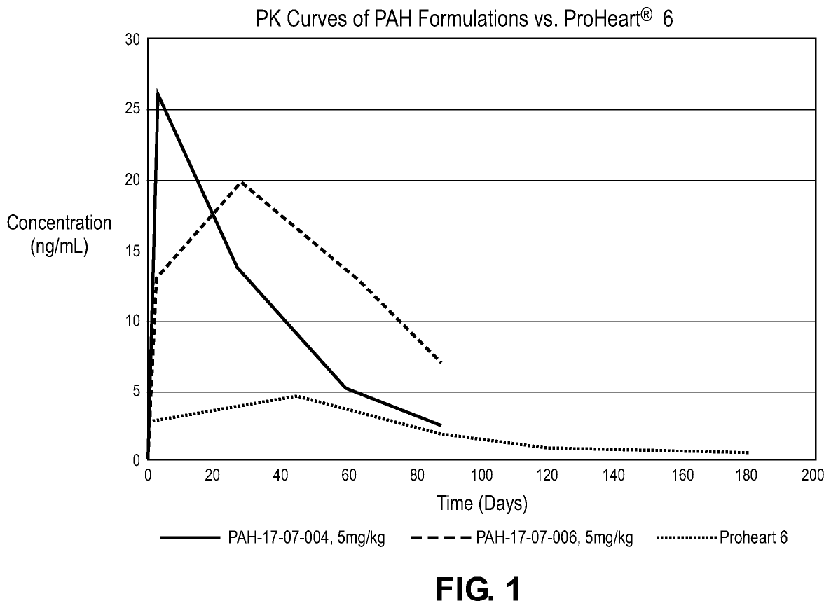

The formulations of Table II (PAH 17-07-0004 of FIG. 1) and Table IV (PAH 17-07-006 of FIG. 1) were prepared for topical administration to canines. 18 dogs were dosed by topical application (6 each for ProHeart®, the formulation of Table II and the formulation of Table IV) at a dosing concentration of about 5 mg/kg. Blood concentrations of moxidectin were present at clinically significant levels (above about 2.5 and 5 ng/ml) for more than 80 days following administration of the composition, as shown in FIG. 1 (dashed and solid lines). Both formulations exhibited higher plasma levels of moxidectin over the treatment period as opposed to ProHeart® which is a subcutaneously injected formulation including moxidectin (dotted line).

### Example II

### Formulations and Pharmacokinetics

The formulation of Table VI was prepared for topical administration to canines. It is expected that blood concentrations of moxidectin in dogs dosed by topical application at a dosing concentration of about 5 mg/kg will be present at clinically significant levels (above about 2.5 and 5 ng/ml) for more than 80 days following administration of the composition.

Although the objects of the disclosure have been described with reference to the above example, it will be understood that modifications and variations are encompassed within the spirit and scope of the disclosure. Accordingly, the disclosure is limited only by the following claims.

1. A topical pharmaceutically acceptable composition, comprising:
a) a pharmaceutically active agent;
b) benzyl alcohol; and
c) propylene carbonate.

2. The composition of claim 1, wherein the benzyl alcohol is present at about 50.0 to 90.0% w/w.

3. The composition of claim 1, wherein the propylene carbonate is present at about 5.0 to 30.0% w/w.

4. A topical pharmaceutically acceptable composition, comprising:
a) a pharmaceutically active agent;
b) N-methyl-2-pyrrolidone (NMP);
c) 2-pyrrolidone; and
d) a triglyceride carrier.

5. The composition of claim 4, wherein the combination of NMP and 2-pyrrolidone are present at about 30.0 to 70.0% w/w.

6. The composition of claim 5, wherein the NMP is present at about 15.0 to 25.0% w/w.

7. The composition of claim 5, wherein the 2-pyrrolidone is present at about 25.0 to 35.0% w/w.

8. The composition of claim 4, wherein the triglyceride carrier is present at about 25.0 to 60.0% w/w.

9. The composition of claim 1, wherein the composition further comprises an antioxidant.

10. The composition of claim 1, wherein the butylated hydroxy toluene (BHT).

11. The composition of claim 1, wherein the pharmaceutically active agent is a macrolide parasiticide.

12. The composition of claim 1, wherein the macrolide parasiticide is moxidectin, selamectin, milbemycin, ivermectin, doramectin, emamectin, eprinomectin, doximectin, abimectin, roxithromycin, clarithromycin, tulathromycin, gamithromycin, dirithromycin, fidaxomicin, megalomicin, erythromycin, azithromycin, or combination thereof.

13. The composition of claim 1, wherein the macrolide parasiticide is moxidectin.

14. The composition of claim 1, wherein the pharmaceutically active agent is an isoxazoline compound.

15. The composition of claim 1, wherein the isoxazoline compound is afoxolaner, fluralaner, sarolaner, lotilaner, or a combination thereof.

16. The composition of claim 1, wherein the composition comprises moxidectin and fluralaner.

17. The composition of claim 1, wherein the pharmaceutically active agent is present in an amount of about 1.0 to 25.0% w/w.

18. The composition of claim 1, wherein at least about 5-15 ng/ml of the pharmaceutically active agent is present in the blood stream of the subject for at least about 50 or 60 days or greater upon administration to a mammal.

19. The composition of claim 1, wherein at least about 2-10 ng/ml of the pharmaceutically active agent is present in the blood stream of the subject for at least about 90 days or greater upon administration to a mammal.

20. A method of treating a disease or disorder in a subject, comprising topically administering to the subject an effective amount of a composition of claim 1.

21. The method of claim 20, wherein the subject is a mammal.

22. The method of claim 20, wherein the subject is a canine.

23. The method of claim 20, wherein the subject is a feline.

24. The method of claim 20, wherein the disease or disorder is an infection.

25. The method of claim 20, wherein the disease or disorder is a parasitic or microbial infection.

26. The method of claim 20, wherein the disease or disorder is ectoparasitic infestation.

27. The method of claim 20, wherein the ectoparasite is body lice, crab lice, scabies, fleas or ticks.

28. The method of claim 20, wherein the parasitic infection is heartworm.

29. The method of claim 20, wherein at least about 5-15 ng/ml of the pharmaceutically active agent is present in the blood stream of the subject for at least about 50 or 60 days or greater upon administration to a mammal.

30. The method of claim 20, wherein at least about 2-10 ng/ml of the pharmaceutically active agent is present in the blood stream of the subject for at least about 90 days or greater upon administration to a mammal.

31. The method of claim 20, wherein the composition is administered once every 2, 3, 4, 5 or 6 months.

32. The method of claim 20, wherein the composition is administered at most once every 2, 3, 4, 5 or 6 months, and wherein the subject remains substantially infection free for at least 2, 3, 4, 5, 6 months or more after each administration.

33. A method of preventing or treating heartworm in a subject, comprising topically administering to the subject an effective amount of a composition of claim 1.

34. The method of claim 33, wherein the subject is a canine or feline.

35. The method of claim 33, wherein the composition is administered at most once every 2, 3, 4, 5 or 6 months, and wherein the subject remains substantially heartworm free for at least 2, 3, 4, 5, 6 months or more after each administration.

36. A method of killing ectoparasites on a subject, comprising topically administering to the subject an effective amount of a composition of claim 1.

37. The method of claim 36, wherein the subject is a canine or feline.

38. The method of claim 36, wherein the ectoparasite is body lice, crab lice, scabies, fleas or ticks.

39. The method of claim 36, wherein the composition is administered at most once every 2, 3, 4, 5 or 6 months, and wherein the subject remains substantially free of living ectoparasites for at least 2, 3, 4, 5, 6 months or more after each administration.

40. A method of killing ectoparasites on a subject and preventing or treating heartworm in the subject, comprising topically administering to the subject an effective amount of a composition of claim 1.

41. The method of claim 40, wherein the subject is a canine or feline.

42. The method of claim 40, wherein the ectoparasite is body lice, crab lice, scabies, fleas or ticks.

43. The method of claim 40, wherein the composition is administered at most once every 2, 3, 4, 5 or 6 months, and wherein the subject remains substantially heartworm free and free of living ectoparasites for at least 2, 3, 4, 5, 6 months or more after each administration.
